(19) United States
(12) Patent Application Publication
(10) Pub. No.: US 2002/0182201 A1
(43) Pub. Date: Dec. 5, 2002

(54) Matrix stabilized enzyme crystals and methods of use

(76) Inventors: Guiyi Zhang-Sun, Potomac, MD (US); Hongsheng Su, Beaconsfield (CA); Xuefeng Yu, Richmond Hills (CA)

Correspondence Address: Maria L. Maebius, Esq., Hale and Dorr LLP, 1455 Pennsylvania Avenue, NW, Washington, DC, 20004, US

(21) Appl. No.: 10/075,542

(22) Filed: Feb. 15, 2002

Related U.S. Application Data
(60) Provisional application No. 60/315,129, filed on Aug. 27, 2001.
(60) Provisional application No. 60/269,316, filed on Feb. 16, 2001.

(51) Int. Cl. A61K 38/46; A61K 38/53; C12N 9/20; C12N 9/64; C12N 9/88

(52) U.S. Cl. 424/94.5; 424/94.6; 435/198; 435/226; 525/54.1; 435/232

(57) ABSTRACT

The invention relates to the stabilization of enzymes through the formation of matrix stabilized enzyme crystals. A matrix stabilized enzyme crystal is formed through cross-linking a polymer having one or more reactive moieties with an enzyme crystal using a low concentration of a multi-functional cross-linking reagent. The invention includes matrix stabilized enzyme crystals of phenylalanine ammonia lyase and a method of using to treat hyperphenylalaninemia.

### CROSS-REFERENCE TO RELATED APPLICATIONS

This application claims the benefit of priority to provisional application Serial No. 60/315,129 filed Aug. 27, 2001, and provisional application Serial No. 60/269,316 filed Feb. 16, 2001, both of which are herein incorporated by reference.

### BACKGROUND OF THE INVENTION

Several methodologies have been developed for stabilizing a protein or enzyme structure while preserving its biological activity. One method involves the internal cross-linking polylyside or other similar polymers with enzyme crystals with a bi-functional chemical reagent. The product of such a reaction is commonly referred to as a cross-linked enzyme crystal or “CLEC” (Margolin, A. L. Trends Biotechnol. 14, 223-230, 1986). The activity of certain enzymes is substantially reduced in the presence of very low levels of cross-linking agents such as glutaraldehyde. The CLEC method is most successful with enzymes which are not sensitive to bi-functional cross-linking reagents, such as glutaraldehyde, thereby allowing its use in high concentrations, 2%-23% (W/V). U.S. Pat. No. 5,618,719 describes the preparation of CLEC-pancreatic elastase using 5% glutaraldehyde; CLEC-pig liver esterase using 11.8% glutaraldehyde, CLEC-lipase from Geotrichum candium using 12.5% glutaraldehyde; CLEC-hen egg lysozyme using 23% glutaraldehyde, CLEC-asparaginase using 7.5% glutaraldehyde; CLEC-Jack Bean urease using 2% glutaraldehyde, CLEC-lipase from Candida cylindracea using 7.5% glutaraldehyde. The CLEC method also was used to prepare CLEC-thermolysin using 12.5% glutaraldehyde (St. Clair and Navia, J. Am. Chem. Soc. 1992, 114, 7314-7316). Additionally, many, but not all enzyme formulations are stable when subjected to the CLEC method in that they retain 30% to 100% of their original catalytic activities and remain active when exposed to elevated temperatures, organic solvents, and/or proteases.

In the CLEC method, reactive side-chain groups of amino acid residues, like the γ-amino group of lysine, are linked to other reactive side-chain groups either in the same molecule or from nearby molecules using a bi-functional cross-linking reagent thereby stabilizing the secondary and tertiary structures of an enzyme and retaining its activity. Internal chemical cross-linking can protect the enzyme crystals from protease degradation by either modifying the protease sensitive sites or by rendering such sites inaccessible to protease degradation. The cross-linked enzyme crystals can retain biological activity provided their substrates are small and can still pass through the channels between protein molecules and that individual enzyme molecules retain sufficient flexibility so that substrate binding and activation can still occur. However, if the active side-chain groups which are chemically cross-linked are near or at an enzyme's active site(s), the cross-linked enzyme may lose some or all of its enzymatic activity.

The stabilization of enzymes, such that they become resistant to protease degradation in the gut, would have significant applicability to treatment of unwanted and/or toxic molecules in the intestine as such stabilized enzymes could be administered orally. For example, hyperphenylalaninemia, which may be defined as a plasma level of phenylalanine of more than 120 umol/L, is a hereditary disease caused by a deficiency in the hepatic enzyme phenylalanine hydroxylase or (in rare cases) its cofactor tetrahydropteria or the cofactor-regenerating enzyme dihydropterin reductase. The disease exists in different forms, phenylketonuria (PKU) which, if the patient is on a normal diet, has plasma phenylalanine levels of more than 1200 umol/L (also measured as 10-20 fold elevated serum phenylalanine levels), and non-PKU hyperphenylalaninemia which has lower levels of plasma phenylalanine. In each form of the disease, the high plasma level of phenylalanine result from failure of the body to successfully catalyze the conversion of the essential amino acid nutrient phenylalanine to tyrosine.

In infancy, sustained increases in plasma levels of phenylalanine greater than 600 umol/L result in mental retardation. The effect appears to be ascribable to phenylalanine itself (not any metabolites thereof), but the mechanism is not yet fully understood. The negative effects of increased plasma levels of phenylalanine may, to a large extent, be prevented if a low-phenylalanine diet is introduced shortly after birth and continued well into adolescence, perhaps for life. Unfortunately, the dietary therapy can be associated with deficiencies of several nutrients, some of which may be detrimental to brain development, and most low phenylalanine products have organoleptic properties that are sufficiently unsatisfactory such that compliance with the dietary treatment is compromised. In addition, pregnant hyperphenylalaninemic patients are required to go back on a strict low-phenylalanine diet in order to avoid the effects of excessive intrauterine phenylalanine, i.e. congenital malformation, microcephaly and mental retardation of the fetus. As recently issued by the NIH, the goal of dietary treatment should be to obtain levels of 2-6 mg/dL during pregnancy, 2-6 mg/dL for neonates through 12 years of age and 2-15 mg/dL after 12 years of age. The strict low-phenylalanine regimen is tiresome for the patients and their families and is very difficult to enforce beyond childhood. Enzyme therapy to make up for the phenylalanine hydroxylase deficiency would therefore provide a great improvement in the treatment of hyperphenylalaninemia.

It has previously been suggested to use phenylalanine ammonia lyase (“PAL”) for treatment of hyperphenylalaninemia, see for instance, Hoskins et al., Lancet, Feb 23, 392-394,1980. Unlike phenylalanine hydroxylase, the phenylalanine-degrading enzyme PAL requires no cofactors to be active. PAL converts phenylalanine to trans-cinnamic acid which, via coenzyme A, is converted to benzoic acid which reacts with glycine and is then excreted via urine primarily as hippurate. PAL may, for instance, be obtained from the yeast Rhodotorula glutinis (also known as Rhodosporidium toruloides), and may also be obtained through recombinant expression of such gene (Sarkissian, et al., Proc. Nat. Acad. Sci., USA, 96:2339-2344, 1999).

Proteolytic degradation of PAL in the gastrointestinal tract has been recognized, e.g., by Gilbert and Jack, Biochem, J. 199, 715-723, 1981, and various attempts to overcome this problem have been published. Such proposals include microencapsulation of the enzyme in “artificial cells” composed of PAL mixed with hemoglobin and enclosed in microspheres covered by a cellulose nitrate membrane (Bourget and Chang, Biochim. Biophys. Acta 883, 432-438, 1986), permeabilised cells of Rhodosporidium toruloides containing the enzyme (H. J. Gilbert and M. Tully, Biochem. Biophys. Res. Comm. 131(2), 1985, pp. 557-563), and PAL that has been cross-linked in permeabilised Rhodotorula cells (Eigtved et al., U.S. Pat. No. 5,753,487).

Numerous attempts by the applicants to generate a stable, active and protease resistant CLEC-PAL formulation have been unsuccessful, due at least in part to the internal chemical cross-linking which occurs in the CLEC method. If an active, stabilized formulation of PAL could be prepared, presumably it could be administered orally to degrade phenylalanine in the gut of hyperphenylalaninemic patients, thereby reducing or maintaining low phenylalanine levels and preventing the serious side effects caused by high phenylalanine levels found in patients suffering from this genetic disorder. Thus there exists a need for an alternate method of preparing stabilized, active enzymes such as PAL and other enzymes which are inactivated by the internal chemical cross-linking of the CLEC method, and applicants have identified such a method.

### SUMMARY OF THE INVENTION

In a first embodiment, the invention is a method for forming matrix stabilized enzyme crystals comprising the step of cross-linking polylyside or other similar polymers with enzyme crystals using a low concentration of multi-functional cross-linking reagent to form an external cross-linked matrix surrounding the enzyme crystal.

In one preferred embodiment, the multi-functional cross-linking reagent is a dialdehyde cross-linking reagent. Suitable dialdehyde cross-linking reagents include both linear and branched dialdehydes. Suitable linear dialdehydes include, without limitation, glutaraldehyde (1,5-Pentanedial), malonaldehyde (1,3-Propanedial), succinaldehyde (1,4-Butanedial), adipaldehyde (1,6-Hexanedial), pimelaldehyde (1,7-Heptanedial), and numerous other linear dialdehydes as would be understood by one of ordinary skill in the chemical arts. Suitable branched dialdehydes include, without limitation, dialdehydes having at least one substituent selected from the group consisting of linear or branched C₁-C₅, —OR₁, wherein R₁ is C₁-C₅, oxygen, nitrogen, sulfur, amino, halogen, and phenyl, such as 3,3-dimethylglutaraldehyde, 3,3-diphenylglutaraldehyde, 3,3-(4-methoxyphenyl)glutaraldehyde, 3-ethyl-2-methyl-1,5-pentanedial, 2-ethyl-3-propyl-1 ,5-pentanedial, 3-ethyl-2,4-dimethyl-1 ,5-pentanedial, 2-ethyl -4-methyl-3-propyl-1,5-pentanedial, 3,4-diethyl-2-methyl-1,5-pentanedial, 3-ethyl-2,4,4′-trimethyl -1,5-pentanedial, 2-ethyl-4,4′-dimethyl-3-propyl -1,5-pentanedial, 2-methyl-2′-propyl-1,5-pentanedial, 3-ethyl-2,4-dimethyl-4′-propyl-1,5-pentanedial, 2-ethyl-4-methyl-3,4′-dipropyl-1,5-pentanedial, 2-butyl-2′-ethyl-1,5-pentanedial, 4-butyl-3,4-diethyl-2-methyl-1,5-pentanedial, 4-butyl-2,4′-diethyl-3-propyl-1,5-pentanedial, 4-methyl pentanedial, aspartaldehyde, 3-(formylmethyl)hexanedial, and numerous other branched dialdehydes as would be understood by one of ordinary skill in the chemical arts.

In another preferred embodiment, the low concentration of the multi-functional cross-linking reagent is a percent concentration of less than 2%, more preferably 0.5% or less, and most preferably 0.2% or less.

In another preferred embodiment, polymers having one or more reactive moieties effective to adhere to the crystal layer include, without limitation, polyamino acids, polycarbohydrates, polystyrenes, polyacids, polyols, polyvinyls, polyesters, polyurethanes, polyolefins, polyethers, and other polymers as would be understood by one of ordinary skill in the chemical arts. Preferably, the polymer is a polyamino acid, and more preferably, a cationic polyamino acid. Suitable polyamino acids include, without limitation, polylysine, polyamides, polyglutamic acids, polyaspartic acids, copolymers of lysine and alanine, copolymers of lysine and phenylalanine, and mixtures thereof. In a most preferred embodiment, the polymer is polylysine.

In a second embodiment, the invention comprises a matrix stabilized enzyme crystal of PAL comprising crystalline PAL cross-linked with a low concentration of multi-functional cross-linking agent in the presence of polylyside.

In one preferred embodiment, the multi-functional cross-linking reagent is a dialdehyde cross-linking reagent. Suitable dialdehyde cross-linking reagents include both linear and branched dialdehydes. Suitable linear dialdehydes include, without limitation, glutaraldehyde (1,5-Pentanedial), malonaldehyde (1,3-Propanedial), succinaldehyde (1,4-Butanedial), adipaldehyde (1,6-Hexanedial), pimelaldehyde (1,7-Heptandial), and numerous other linear dialdehydes as would be understood by one of ordinary skill in the chemical arts. Suitable branched dialdehydes include, without limitation, dialdehydes having at least one substituent selected from the group consisting of linear or branched C₁-C₅, —OR₁, wherein R₁ is C₁-C₅, oxygen, nitrogen, sulfur, amino, halogen, and phenyl, such as 3,3-dimethylglutaraldehyde, 3,3-diphenylglutaraldehyde, 3,3-(4-methoxyphenyl)glutaraldehyde, 3-ethyl-2-methyl-1,5-pentanedial, 2-ethyl-3-propyl-1,5-pentanedial, 3-ethyl-2,4-dimethyl-1,5-pentanedial, 2-ethyl -4-methyl-3-propyl-1,5-pentanedial, 3,4-diethyl-2-methyl-1,5-pentanedial, 3-ethyl-2,4,4′-trimethyl -1,5-pentanedial, 2-ethyl-4,4′-dimethyl-3-propyl -1,5-pentanedial, 2-methyl-2′-propyl-1,5-pentanedial, 3-ethyl-2,4-dimethyl-4′-propyl-1,5-pentanedial, 2-ethyl-4-methyl-3,4′-dipropyl-1,5-pentanedial, 2-butyl-2′-ethyl-1,5-pentanedial, 4-butyl-3,4-diethyl-2-methyl-1,5-pentanedial, 4-butyl-2,4′-diethyl-3-propyl-1,5-pentanedial, 4-methyl pentanedial, aspartaldehyde, 3-(formylmethyl)hexanedial, and numerous other branched dialdehydes as would be understood by one of ordinary skill in the chemical arts.

In another preferred embodiment, the low concentration of the multi-functional cross-linking reagent is a percent concentration of less than 2%, more preferably 0.5% or less, and most preferably 0.2% or less.

In another preferred embodiment, polymers having one or more reactive moieties effective to adhere to the crystal layer include, without limitation, polyamino acids, polycarbohydrates, polystyrenes, polyacids, polyols, polyvinyls, polyesters, polyurethanes, polyolefins, polyethers, and other polymers as would be understood by one of ordinary skill in the chemical arts. Preferably, the polymer is a polyamino acid, and more preferably, a cationic polyamino acid. Suitable polyamino acids include, without limitation, polylysine, polyamides, polyglutamic acids, polyaspartic acids, copolymers of lysine and alanine, copolymers of lysine and phenylalanine, and mixtures thereof. In a most preferred embodiment, the polymer is polylysine.

In a third embodiment, the invention is a method of treating a hyperphenylalaninemic patient comprising administering a therapeutically effective amount of matrix stabilized enzyme crystals of PAL. In a preferred embodiment, the administration is oral.

### BRIEF DESCRIPTION OF THE DRAWINGS

FIG. 1 shows the stability and retention of PAL activity for MSEC-PAL when contacted with pronase or mouse intestinal fluid.

FIG. 2 shows the distribution of recovered phenylalanine degrading activity following oral administration of MSEC-PAL.

FIG. 3 shows a decrease in plasma phenylalanine following oral administration of MSEC-PAL. The x-axis shows time, in hours, elapsed after subcutaneous injection of phenylalanine. The y-axis shows the phenylalanine concentration in the plasma as determined from blood samples.

### DETAILED DESCRIPTION OF THE INVENTION

Applicants have discovered a method of preparing active, stable and protease resistant enzyme crystals in which the enzymes are not extensively cross-linked as in the CLEC method. The stabilized enzyme crystal of applicants' invention is termed herein a “matrix stabilized enzyme crystal” and is useful for the treatment of unwanted and/or toxic molecules in the intestines of animals, including humans and other mammals.

In the method of the invention, the enzyme crystals are cross-linked in the presence of a very low concentration of a multi-functional cross-linking reagent. Specifically, less than 2%, preferably 0.5% or less, and most preferably, 0.2% or less (W/V) of multi-functional cross-linking reagent (such as glutaraldehyde) is used, as opposed to the 2-23% (W/V) of glutaraldehyde which is used in the CLEC method. Such reduced amounts of multi-functional cross-linking reagent would typically be ineffective in the CLEC method. However, the higher glutaraldehyde concentrations required by the CLEC method are sufficient to inactivate the activity of certain enzymes such as PAL.

In one preferred embodiment, the multi-functional cross-linking reagent is a dialdehyde cross-linking reagent. Suitable dialdehyde cross-linking reagents include both linear and branched dialdehydes. Suitable linear dialdehydes include, without limitation, glutaraldehyde (1,5-Pentanedial), malonaldehyde (1,3-Propanedial), succinaldehyde (1,4-Butanedial), adipaldehyde (1,6-Hexanedial), pimelaldehyde (1,7-Heptanedial), and numerous other linear dialdehydes as would be understood by one of ordinary skill in the chemical arts. Suitable branched dialdehydes include, without limitation, dialdehydes having at least one substituent selected from the group consisting of linear or branched C₁-C₅, —OR₁, wherein R₁ is C₁-C₅, oxygen, nitrogen, sulfur, amino, halogen, and phenyl, such as 3,3-dimethylglutaraldehyde, 3,3-diphenylglutaraldehyde, 3,3-(4-methoxyphenyl)glutaraldehyde, 3-ethyl-2-methyl-1,5-pentanedial, 2-ethyl-3-propyl-1,5-pentanedial, 3-ethyl-2,4-dimethyl-1,5-pentanedial, 2-ethyl -4-methyl-3-propyl-1,5-pentanedial, 3,4-diethyl-2-methyl-1,5-pentanedial, 3-ethyl-2,4,4′-trimethyl -1,5-pentanedial, 2-ethyl-4,4′-dimethyl-3-propyl -1,5-pentanedial, 2-methyl-2′-propyl-1,5-pentanedial, 3-ethyl-2,4-dimethyl-4′-propyl-1 ,5-pentanedial, 2-ethyl-4-methyl-3,4′-dipropyl-1,5-pentanedial, 2-butyl-2′-ethyl-1,5-pentanedial, 4-butyl-3,4-diethyl-2-methyl-1,5-pentanedial, 4-butyl-2,4′-diethyl-3-propyl-1,5-pentanedial, 4-methyl pentanedial, aspartaldehyde, 3-(formylmethyl)hexanedial, and numerous other branched dialdehydes as would be understood by one of ordinary skill in the chemical arts.

In addition to a low concentration of a multi-functional cross-linking reagent, one or more polymers having one or more reactive moieties is used in conjunction with the cross-linking reagent to form a net-like structure on the crystal surface. Suitable polymers include cationic, anionic, and/or hydrophobic polymers having an average molecular weight of between about 100 and about 1,000,000, preferably between about 200 and about 750,000, and most preferably between about 500 and about 150,000. The reactive moieties can include electrophilic and/or nucleophilic groups such as, for example, haloalkyls, epoxides, hydrazides, hydrazines, thiolates, hydroxyls, and the like, preferably active esters, amines, carboxylic acids, sulfhydryls, carbonyls, and carbohydrates. The reactive moieties along the length of the polymer adhere to the surface of the crystal forming the net-like structure when cross-linked with the cross-linking reagent. For example, a cationic reactive moiety may become attached to the surface of the crystals by a charge-charge interaction. Useful polymers include homopolymers having a single repeating monomer unit, copolymers having two different monomer units, or polymers having more than two different monomer units. Preferably, homopolymers or copolymers are used.

Suitable polymers having one or more reactive moieties effective to adhere to a crystal layer preferably include polyamino acids, including homopolymers, such as polylysine, polyamides, polyglutamic acid, polyaspartic acid, polycarbohydrates, polystyrenes, polyacids, polyols, polyvinyls, polyesters, polyurethanes, polyolefins, polyethers, and the like. The mentioned polymers have a cationic reactive moiety, but other anionic and/or hydrophobic polyamino acids are useful. The polyamino acid could also be a copolymer. When a copolyamino acid is used, at least two amino acid residues are present in the polymer provided that the full length polymer comprises reactive moieties effective to form the net-like structure on the crystal surface. The ratio of one amino acid to another in a copolyamino acid having one or more reactive moieties can be from about 0.1 to about 100 of one amino acid per unit of the other amino acid. Preferably, the ratio is 1:1. Suitable amino acids for forming copolyamino acids include any combinations of the following amino acids: lysine, alanine, phenylalanine, serine, tryptophan, cysteine, histidine, arginine, glycine, glutamine, proline, leucine, isoleucine, threonine, asparagine, valine, methionine, tyrosine, aspartic acid, and/or glutamic acid. Preferably, the polymer is a polyamino acid, such as the homopolymer polylysine, the copolymer of lysine and alanine in a 1:1 ratio, or the copolymer of lysine and phenylalanine in a 1:1 ratio. In another preferred embodiment, the polyamino acid is cationic. The homopolymer polylysine is the most preferred polymer.

Other suitable polymers having one or more reactive moieties effective to adhere to a crystal layer include, for example, polycarbohydrates and polysaccharides such as, for example, polyamylose, polyfuranosides, polypyranosides, carboxymethylamylose, and dextrans; polystyrenes such as, for example, chloromethylated polystyrene and bromomethylated polystyrene; polyacrylamides such as, for example, polyacrylamide hydrazide; polyacids such as, for example, polyacrylic acid; polyols such as, for example, polyvinyl alcohol; polyvinyls such as, for example, polyvinyl chloride and polyvinyl bromide; polyesters; polyurethanes; polyolefins; and polyethers.

The function of the one or more reactive moieties of the polymer provides that the reactive moieties interact with the cross-linking reagent at the surface of the enzyme crystals without reacting with the internal amino acid residues of the enzyme crystals. The reactive side groups of the polymer may be cross-linked by multi-functional coupling reagents. By way of example, in the case of polylysine, the net-like structure on the crystal surfaces is formed by having the polylyside polymers cross-link to each other, and/or with amine groups of the lysine residues on the crystals' surface. Due to the use of low concentrations of the multi-functional cross-linking reagent, those amine groups which are readily accessible, in particular, those provided by the polylysine or those which are on the surface of the enzyme crystals, react preferentially while the amino groups of internal amino acid residues of the enzyme crystals, such as lysine, have an insignificant or no opportunity to react with the cross-linking reagent and their ability to participate in enzymatic reactions is maintained. In addition, the molecular size and/or the charge of the added polymer having reactive side groups may prevent penetration of the multi-functional cross-linking reagent into and through the channels of the enzyme crystals, and thus the internal lysine residues of the enzyme are not significantly cross-linked to each other or between the enzymes in the crystal.

The matrix formed by the cross-linking of polylyside on the surface of an enzyme crystal maintains the enzyme crystal's physical structure and provides mechanical and thermal stability as well as the protection against proteases. The matrix stabilized enzyme crystals also remain permeable to small substrate molecules as shown by the retention of their enzymatic activity as discussed further below. Since the method of the invention for forming matrix stabilized enzyme crystals involves surface modification rather than internal cross-linking, it can be used in all enzymes including glutaraldehyde-sensitive ones. Suitable enzymes for use in the present invention include those enzymes that for which the CLEC method is applicable and particularly, crystalline enzymes. Without limitation, for example, phenylalanine ammonia lyase, L-methionine-γ-lyase, lipases, carboxypeptidase-A, are suitable for use in the present invention.

Applicants have successfully applied the method of the invention to prepare stable, active and protease resistant PAL. Using animal models, applicants have demonstrated that matrix stabilized enzyme crystals of PAL (“MSEC-PAL”) are active, stable and protease resistant. Thus a MSEC-PAL may be useful for the oral treatment of hyperphenylalaninemic patients to lower their plasma concentration of phenylalanine.

The method of preparing matrix stabilized enzyme crystals, using PAL as an illustrative example, is described below.

1 . PAL Microcrystal Preparation

In a first step of preparing PAL for stabilization as an enzyme crystal, a method to produce PAL crystals was developed. The PAL used for crystallization may be purified or recombinantly produced according to known techniques.

Briefly, an aqueous solution of PEG8000 (50% w/v; filtered through a 0.22 mm Millipore filter) is slowly added to a solution of 20 mg/mL PAL in 25 mM Na₂HPO₄NaH₂PO₄, pH 6.5, with very gentle swirling, to a final PEG8000 concentration of 10% (w/v). A saturated Li₂SO₄ solution (in 50 mM sodium phosphate, pH. 6.5) is slowly added to a final concentration of approx. 15 mM. A fine precipitate which may form during the addition of Li₂SO₄ is removed by centrifugation or filtration. Crystallization of PAL is initiated by the continued addition of Li₂SO₄ to a final concentration of approx. 30 mM. The resulting solution is then stored at 4° C. During an overnight incubation at 4° C., large amounts of rod-shaped microcrystals of a size of 10-70 microns form and settle to the bottom of the container.

The crystallization of PAL can be accelerated by adding a few previously formed PAL microcrystals to the PAL-10% PEG8000-Li₂SO₄ solution. Using previously prepared PAL microcrystals as seeds, new PAL microcrystals can form after 30 minutes of incubation at 4° C.

2. Formation of CLEC-PAL

In the CLEC method enzyme crystals in solutions of various compositions and pH values between 5 and 7 are cross-linked with the bi-functional cross-linking agent glutaraldehyde at final concentrations between 2% (w/v) and 24% (w/v). As a preliminary investigation, applicants assessed the capability of the CLEC method to prepare stable, active preparations of PAL, to be used as a control in their development of an alternate enzyme stabilization method. In a typical experiment, rod shaped PAL micro-crystals, produced as described above were used. PAL micro-crystals in 20 mM sodium phosphate, 15% PEG 6000, 18 mM LiSO4 at pH 7.0 were incubated at 25° C. during 40 min. with glutaraldehyde at concentrations ranging from 0.05% to 2.5% (w/v). The cross-linking reaction was stopped by replacing the reaction solution with 100 mM Tris pH.8.5 after a short centrifugation of the cross-linked crystals. To assess the resistance of those CLEC-PAL micro-crystals that had retained some activity against proteolytic enzymes (at glutaraldehyde concentrations <0.2%), the CLEC-PAL micro-crystals were incubated for 30 min. at 37° C. after addition of diluted mouse small intestinal fluid.

Results (Table 1)

As shown in Table 1, all activity was lost in the CLEC-PAL micro-crystals produced according to the CLEC method using glutaraldehyde concentrations greater than 0.23%.

The activity of CLEC-PAL micro-crystals, produced according to the CLEC method, was almost completely lost, at glutaraldehyde concentrations below 0.2% . At these gluteraldehyde concentrations the CLEC-PAL microcrystals were degraded when exposed to proteases.

Glutaraldehyde concentrations below 0.1% resulted in CLEC-PAL micro-crystals which were not stable and dissolved in 100 mM Tris at pH 8.5.

TABLE 1
Effect of glutaraldehyde on PAL activity

| Percent | | Degraded |
|---|---|---|
| Glutaraldehyde | Activity of CLEC-PAL(IU) | by Proteases |
| 0.0% | 60 (before Cross-linking | NA |
| 0.05% | Crystals dissolve in buffer | NA |
| 0.1% | 11.0 | yes |
| 0.14% | 5.0 | yes |
| 0.19% | 2.4 | yes |
| 0.23% | 1.5 | yes |
| 0.33% | 0.0 | Not tested |
| 0.45% | 0.0 | Not tested |
| 0.65% | 0.1 | Not tested |
| 1.43% | 0.2 | Not tested |
| 1.89% | 0.2 | Not tested |
| 2.5% | 0.1 | Not tested |

All attempts to produce CLEC-PAL micro-crystals under various reaction conditions, including the use of cross-linking reagents other than glutaraldehyde, were unsuccessful. No reaction conditions could be found that resulted in CLEC-PAL micro-crystals which at the same time, both retained significant PAL activity and were stable against proteolytic degradation. These results suggested that one or more critical lysine residue(s) or other reactive amino acid residues in PAL may be located at or near the catalytic site and reaction of glutaraldehyde with the residue(s) results in inactivation of the enzyme. The applicants have concluded that PAL micro-crystals obtained from a PEG and phosphate buffer solution could not be cross-linked using a glutaraldehyde to form a stable and functional PAL micro-crystal formulation, thus the CLEC method was not successful for PAL.

It is known that enzymes other than PAL also contain lysine residues that are sensitive to multi-functional cross-linking reagents, and thus are not suitable for stabilization using the CLEC method. One example is the enzyme Carboxypeptidase-A which is 300 times less active after cross-linking with 1% (W/V) glutaraldehyde, (Quiocho and Richards, Biochemistry, Vol. 5 4062-4076, 1966). Hence, an alternative enzyme stabilization method that can be used for all enzyme crystals is needed.

3. Formation of Matrix Stabilized Enzyme Crystals

Rod shaped PAL micro-crystals formed as described above were cross-linked in the presence of polylysine using a low concentration of glutaraldehyde. Specifically, MSEC-PAL was prepared as follows: The supernatant is removed from the precipitated crystals and replaced with fresh buffer containing 20% (w/v) PEG8000, 20 mM Li₂SO₄ and 10 mM sodium phosphate. The volume of buffer added was adjusted to obtain a PAL activity of between 120 and 130 IU/ml. One hundred mL of this solution of crystals was brought to room temperature (RT), then 3.5 mL of poly-L-Lysine 5000 (50 mg/ml) was added and mixed well. Incubation was at RT for 30 mins, with mixing every 10 mins. Next, 2.48 mL of glutaraldehyde (0.3% v/v in dH₂O) was added and incubated for a further 90 mins, at RT. Finally, 0.5 M Tris, pH 8.5, was added so that the final volume was 200 mL. The PAL activity of the MSEC preparations were measured 30 to 60 mins later.

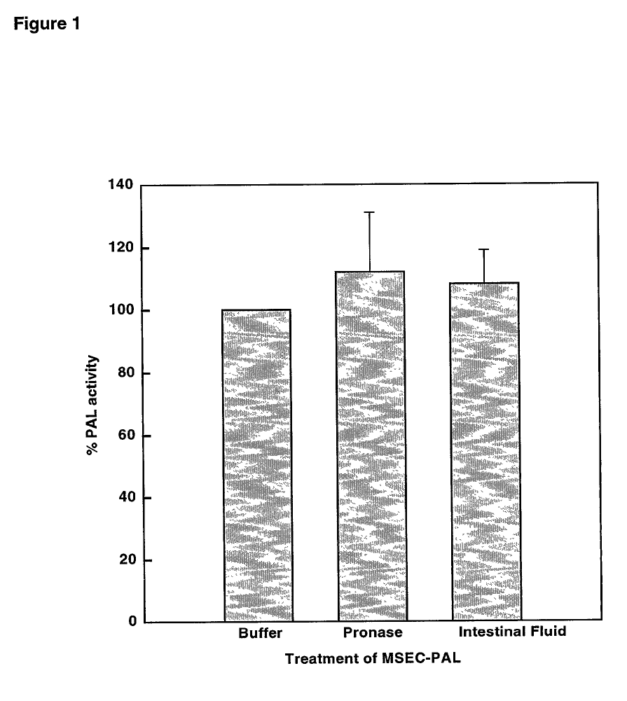

Following preparation of the MSEC-PAL, the PAL enzyme activity of such crystals was determined to be greater than 20% of the solubilized PAL enzyme's original value. To determine if the MSEC-PAL was resistant to proteolytic degradation, two types of experiments were performed (FIG. 1). First, MSEC-PAL was incubated with 10 mg/mL of pronase for 2 hrs at 37° C. The activity of the PAL enzyme was unaffected by exposure to pronase. Second, MSEC-PAL was incubated in mouse intestinal fluid (12.5% v/v) overnight at 37° C. The activity of MSEC-PAL was also unaffected by exposure to intestinal fluid. In comparison, there was no detectable activity in non-crosslinked PAL crystals following incubation with pronase or intestinal fluid. These experiments demonstrate that the MSEC preparations of PAL, retain enzymatic activity and that this activity is protected from degradation by proteases.

The applicants have also used this methodology on another glutaraldehyde sensitive enzyme, methionine-γ-lyase, with similar results. Because the matrix stabilized enzyme crystals method involves surface modification of enzyme crystals, this method should also be applicable for the stabilization of any enzymes which are not suitable for stabilization using the CLEC method.

4. Delivery of Enzymatically-Active MSEC-PAL to the Gastrointestinal Tract of Rats

Twenty Sprague-Dawley rats, ˜2 months old, weighing ˜175-200 grams, were used in this study. The animals were maintained on a regular diet prior to experimentation.

Food was removed 24 hours prior to the experiment and the animals were placed in cages with grid flooring. Sugar water (5%) was introduced as an energy source. All animals were divided into four groups. The four rats in group one were gavaged with 1.5 ml of 0.5M Tris-HCl buffer, while the rats in group two were gavaged with 1.5 ml solution containing 50 IU unformulated PAL in 0.5M Tris-HCl buffer, pH8.5. Group three had eight rats which were gavaged with 1.5 ml of MSEC-PAL (50 IU in 0.5M Tris-HCl buffer, pH8.5). All 16 rats in these three groups were sacrificed 45 minutes following the gavage. The four rats of group four were gavaged with 1.5 ml of MSEC-PAL (50 IU in 0.5M Tris-HCl buffer, pH8.5) and sacrificed 90 minutes following the gavage.

After sacrifice, the stomach and small intestine were immediately removed. The content of the small intestine was flushed with 20 ml of 0.1M Tris-HCl buffer, pH8.5, and the content of the stomach was flushed with 10 ml of 0.1M Tris-HCl buffer, pH8.5. Test-tubes with intestinal or stomach contents were stored on ice.

PAL activity was measured spectrophotometrically. A sample of 50 μl was mixed with 950 μl of assay buffer (22.5 mM L-phenylalanine in 0.1M Tris-HCl buffer, pH8.5) prewarmed at 30° C. The increase in absorbance at 290 nm was monitored at 30° C. One unit of enzyme activity is defined as the amount of enzyme that catalyzes the formation of 1 μmol of trans-cinnamic acid. Assays were performed immediately following collection of samples.

In the four rats in group one gavaged with 1.5 ml of Tris-HCl buffer, no PAL activity was detected in their stomachs or small intestines (Table 2). Similarly, there was no detectable PAL activity in the four animals in group two that were gavaged with 54 IU of unformulated PAL in 0.5 M Tris-HCl buffer.

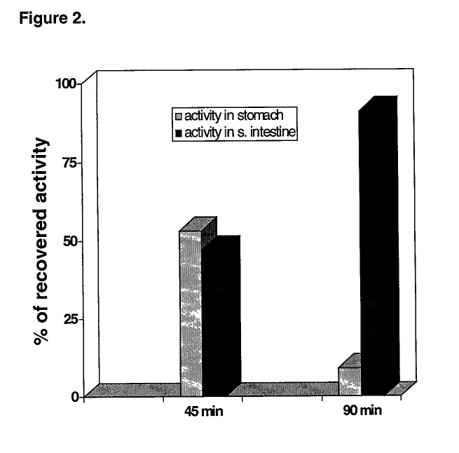

However, PAL activity could be recovered from both rat stomachs and intestines 45 minutes or 90 minutes after gavage with MSEC-PAL (Table 2, FIG. 2), which demonstrates that the MSEC formulation protected the PAL enzyme against protease degradation in the small intestine. When the stomach and intestinal contents were collected 45 minutes after oral administration of the MSEC formulated PAL, the average recovered PAL activity from both the stomach and the small intestine was 17.8 IU (7.1 IU to 23.7 IU) in the eight animals, which was 34.3% (13.1% to 48.1%) of the gavaged activity. About 53% of the recovered activity was still in the stomach, while 47% of the activity was found in the small intestinal content (FIG. 2).

When the stomach and intestinal contents were collected 90 minutes following the oral administration of MSEC-PAL, the average recovered activity from both the stomach and the intestine was 8.1 IU (3.7 IU to 9.3 IU) in the four animals, which was 16.2% (7.4% to 22.0%) of the gavaged activity. Close to 9% of the recovered activity was in the stomach, while over 91% of the activity was found in the small intestinal content (FIG. 2).

TABLE 2
Phenylalanine degrading activity of orally administered MSEC-PAL

Phenylalanine
Degrading Activity (IU)

| Treatment Group | N | Stomach | Small Intestine |
|---|---|---|---|
| 1 Tris-HCl Buffer | 4 | 0.0 ± 0.0 | 0.0 ± 0.1 |
| 2 PAL (unformulated) | 4 | 0.1 ± 0.1 | 0.1 ± 0.2 |
| 3 PAL-MSEC | 8 | 9.5 ± 7.4 | 8.3 ± 4.9 |
| 4 PAL-MSEC | 4 | 0.7 ± 0.2 | 7.4 ± 3.3 |

Values are the mean ±SD for the number of animals indicated. Animals in the first three groups were sacrificed 45 min after oral gavage of the indicated treatment, and the four animals in group 4 were sacrificed 90 minutes after oral gavage.

5. Decrease in Plasma Phenylalanine Following Oral Administration of MSEC-PAL

A hybrid strain of ENU mice (Pah^{enu2/enu2}) was used in these experiments (Sarkissian et. al. (1999) Proc. Natl. Acad. Sci. 96:2339-2344.). This strain of mouse is deficient in hepatic phenylalanine hydroxylase, and has a 10 to 20 fold increase in plasma phenylalanine levels compared to normal mice, when fed a standard diet. Plasma phenylalanine levels in these mice can be lowered to normal values by placing them on a phenylalanine-free diet.

Eight ENU mice, approximately 25 g in weight, were maintained on a normal diet. All mice were then placed on a phenylalanine-free diet (Harlan Teklad diet 2826), and given water containing 30 mg/L of L-phenylalanine for 5 days prior to the start of the experiment.

Part A: On the day of the experiment, animals were given a subcutaneous injection of 150 μg/g (L-phenylalanine/g body weight) at time 0 (t=0 hrs). Four animals were gavaged with 5.5 IU of MSEC-PAL in 0.4 ml of 0.5M Tris-HCl buffer (pH 8.5) at t=1, 2, 3, and 4 hrs. Controls consisted of four mice gavaged with an equal volume of Tris buffer. Blood samples were obtained from the tail vein 5 mins prior to the subcutaneous phenylalanine injection (t=0 hr) and at one hour intervals thereafter (t=1 to 7 hrs.).

Part B: All animals were returned to a normal diet for two days, then given a phenylalanine free diet for 5 days. The second part of this experiment was a cross over study, in which the mice in the treated and control groups were switched, such that mice treated with MSEC-PAL in part A were given buffer only in part B, and mice given buffer in part A were given MSEC-PAL in part B. Mice in part B were otherwise treated as described in part A above.

Plasma phenylalanine levels were determined after centrifugation of heparinized blood samples. Phenylalanine concentrations were measured by high pressure liquid chromatography (HPLC) using the Beckman System Gold, DABS amino acid sampling kit. Results were analyzed by pooling data from part A and B, such that eight mice received oral MSEC-PAL and eight control mice received buffer alone. Data was analyzed using analysis of variance coupled with t-tests to identify differences between treatment groups.

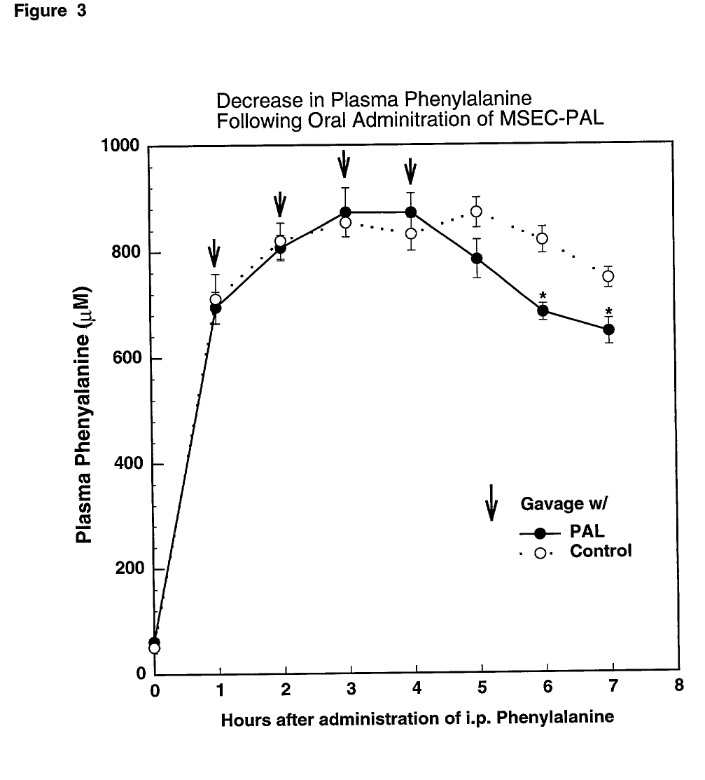

As shown in FIG. 3, the injection of subcutaneous phenylalanine caused a sharp increase in plasma phenylalanine in all mice. Plasma concentrations are expressed in μM, and shown as the mean ±S.E.M, with n=8. There was no difference between the control and treated groups during the gavage period (t=1 to 4 hrs). However, one hour after the last gavage (t=5hrs), plasma phenylalanine levels in MSEC-PAL treated mice decreased in comparison to buffer treated controls. This decrease in plasma phenylalanine was significant at t=6 and 7 hrs, with P<0.001 at 6 hrs and P=0.006at 7hrs.

These results demonstrate that an oral formulation (MSEC-PAL) of phenylalanine lyase can significantly lower the concentration of phenylalanine in the plasma. The observed effects of MSEC-PAL also indicate that this formulation protects enzymes from proteolytic degradation in the gastrointestinal tract.

All publications mentioned in this specification are herein incorporated by reference, to the same extent as if each individual publication was specifically and individually indicated to be incorporated by reference.

It will be understood that the invention is capable of further modifications and this application is intended to cover any variations, uses, or adoptions of the invention including such departures from the present disclosure as come within known or customary practice within the art to which the invention pertains, and is intended to be limited only by the appended claims.

1. A method for forming matrix stabilized enzyme crystals comprising the step of cross-linking a crystalline enzyme with at least one polymer having one or more reactive moieties effective to adhere to the crystal layer of the crystalline enzyme using a multi-functional cross-linking reagent in an amount sufficient to form said matrix stabilized enzyme crystals which are resistant to degradation by proteolytic enzymes.

2. The method of claim 1 wherein the enzyme is selected from the group consisting of phenylalanine ammonia lyase, L-methionine-γ-lyase, lipases, and carboxypeptidase-A.

3. The method of claim 1, wherein the enzyme is phenylalanine ammonia lyase.

4. The method of claim 1 wherein the multi-functional cross-linking reagent is a dialdehyde cross-linking reagent.

5. The method of claim 4 wherein the dialdehyde cross-linking reagent is a linear or branched dialdehyde.

6. The method of claim 4 wherein the dialdehyde cross-linking reagent is selected from the group consisting of substituted or unsubstituted glutaraldehyde (1,5-Pentanedial), malonaldehyde (1,3-Propanedial), succinaldehyde (1,4-Butanedial), adipaldehyde (1,6-Hexanedial), pimelaldehyde (1,7-Heptanedial).

7. The method of claim 4 wherein the dialdehyde cross-linking reagent is glutaraldehyde.

8. The method of claim 1, wherein the multi-functional cross-linking reagent is used in a percent concentration of less than 2% (w/v).

9. The method of claim 8, wherein the multi-functional cross-linking reagent is used in a percent concentration of 0.5% or less (w/v).

10. The method of claim 9, wherein the multi-functional cross-linking reagent is used in a percent concentration of 0.2% or less (w/v).

11. The method of claim 1, wherein the polymer having one or more reactive moieties effective to adhere to the crystal layer is a polyamino acid, a polycarbohydrate, a polystyrene, a polyacid, a polyol, a polyvinyl, a polyester, a polyurethane, a polyolefin, or a polyether.

12. The method of claim 11, wherein the polymer having one or more reactive moieties effective to adhere to the crystal layer is a polyamino acid.

13. The method of claim 12, wherein the polyamino acid is a polylysine, a polyamide, a polyglutamic acid, a polyaspartic acid, a copolymer of lysine and alanine, or a copolymer of lysine and phenylalanine.

14. The method of claim 13, wherein the polyamino acid is polylysine.

15. The method of claim 14, wherein said enzyme is phenylalanine ammonia lyase.

16. The method of claim 14, wherein the multi-functional cross-linking reagent is used in a percent concentration of 0.5% or less (w/v).

17. The method of claim 16, wherein the multi-functional cross-linking reagent is used in a percent concentration of 0.2% or less (w/v).

18. Matrix stabilized enzyme crystals prepared according to the method of claim 1.

19. Matrix stabilized enzyme crystals prepared according to the method of claim 14.

20. Matrix stabilized enzyme crystals prepared according to the method of claim 15.

21. Matrix stabilized enzyme crystals of phenylalanine ammonia lyase comprising crystalline PAL cross-linked with a bifunctional cross-linking agent in the presence of polylysine.

22. The matrix stabilized enzyme crystals of claim 21, wherein said bifunctional cross-linking agent is glutaraldehyde.

23. A method of treating hyperphenylalaninemia comprising administering a therapeutically effective amount of matrix stabilized enzyme crystals of phenylalanine ammonia lyase.

24. The method of claim 23, wherein said matrix stabilized enzyme crystals of phenylalanine ammonia lyase are stabilized by cross-linking polylysine with phenylalanine ammonia lyase in the presence of less than 0.5% w/v bifunctional cross-linking agent.

25. The method of claim 24, wherein said bifunctional cross-linking agent is glutaraldehyde.

26. The method of claim 23, wherein the administration of matrix stabilized enzyme crystals of phenylalanine ammonia lyase is conducted by oral administration.
